(19) United States
(12) Patent Application Publication
(10) Pub. No.: US 2015/0260819 A1
(43) Pub. Date: Sep. 17, 2015

(54) TRANSFER OF VALIDATED CAD TRAINING DATA TO AMENDED MR CONTRAST LEVELS

(71) Applicant: Siemens Aktiengesellschaft, Munich (DE)

(72) Inventors: Lars LAUER, Neunkirchen (DE); Xian Sean ZHOU, Exton, PA (US)

(73) Assignee: SIEMENS MEDICAL SOLUTIONS USA, INC., Malvern, PA (US)

(21) Appl. No.: 14/637,768

(22) Filed: Mar. 4, 2015

(30) Foreign Application Priority Data
Mar. 11, 2014 (DE) 102014204467.7

(51) Int. Cl. G01R 33/56; G01R 33/54; G01R 33/385; A61B 5/055

(52) U.S. Cl. CPC G01R 33/5608; A61B 5/055; G01R 33/543; G01R 33/3854

(57) ABSTRACT

A method is disclosed for controlling an MR device. A control unit and an imaging device with a control unit are also disclosed. An embodiment of the method serves to transfer an interim result which is calculated by an automatic alignment algorithm which is provided in an image processing module, to a localizer image. An optimized result can then be converted into instructions which serve to control the MR device and are based on the localizer image which has been captured in a different recording technique than the images with which the automatic alignment algorithm has been trained.

### PRIORITY STATEMENT

The present application hereby claims priority under 35 U.S.C. §119 to German patent application number DE 102014204467.7 filed Mar. 11, 2014, the entire contents of which are hereby incorporated herein by reference.

### FIELD

At least one embodiment of the present invention generally relates to the field of image processing and medical technology and concerns, in particular, the control of a magnetic resonance device based on prior recordings which are analyzed by the use of image processing algorithms (e.g. CAD algorithms, Computer Aided Diagnosis) and by a set of training data, in order to be able to set the parameters for the planned magnetic resonance tomographic examination (e.g. in the context of an automatic alignment procedure).

### BACKGROUND

In order to be able to determine a particular volume region of the tissue to be investigated in a targeted manner, an overview image (localizer image) is often used. A large number of automated methods for marking and automatically determining an interesting volume region (volume of interest/VOI or region of interest/ROI) in this overview image are known from the prior art. In relatively modern magnetic resonance devices, a mode can also be provided for automatic slice alignment, which is also known as “auto-align mode”. With the automatic alignment mechanism, layer positioning and/or an automatic orientation of the layer position is suggested.

An automatic alignment procedure is known, for example, from DE 102012208325 A1. Herein, automatic positioning and adaptation is described in an adjustment process for a shim field map based on automatic alignment and AutoCoverage procedures.

The automatic alignment mechanism and other automatic algorithms from the field of Computer-Aided Diagnosis are based on training data which have to be confirmed and/or validated in complex clinical test processes.

One problem with the methods conventionally applied in the prior art lies therein that the respective image processing algorithm applied (e.g. the automatic alignment mechanism) is validated only for a particular training data set and can therefore only be utilized for example for the respectively determined contrast level at which the relevant training data set has been acquired. If the CAD tool is to be configured for a new training set (e.g. with a changed contrast or another measuring sequence), it has previously been necessary in the prior art for the training to be carried out again in full in order to create a new training data set for the tool. This process is time-intensive and therefore costly.

### SUMMARY

At least one embodiment of the present invention provides improved possibilities for controlling an MR device. In particular, the transfer of training data which are acquired with particular recording parameters also to such images that have been acquired with other recording parameters is to be made possible. Furthermore, a correspondingly improved control unit for an imaging device, in particular for an MR system, is to be provided.

A method is disclosed for controlling an imaging device, in particular a magnetic resonance device, via a control unit for an imaging device and via an imaging device, in particular a magnetic resonance device.

Features, advantages or alternative embodiments mentioned herein are also to be applied equally to the other claimed subject matter and vice versa. In other words, the present claims (which are directed, for example, to a control unit or an imaging device) can also be further developed with the features disclosed or claimed in connection with the method. The corresponding functional features of the method are configured with suitable modules as contained herein, including in particular hardware modules and/or microprocessor modules.

According to one embodiment, the invention relates to a method for controlling an imaging device by using an image processing procedure on at least one localizer image recorded via the imaging device, comprising the following:

providing an image processing procedure which has been trained in a training phase with first images of a TRAIN image data set that have been acquired using a first recording technique, wherein for each first image, the TRAIN image data set also comprises at least one second image (there can also be a plurality of second images), wherein the second image has been acquired in the same scan but, as distinct from the first image, in each case with a different, second recording technique;

providing the localizer image for use of the image processing procedure wherein the localizer image has been acquired with the second recording technique;

applying the image processing procedure to the first image of the TRAIN image data set for calculating an interim result;

transferring the calculated interim result to the localizer image associated with the second image in order to calculate a result;

using an optimization method on the result; and

converting the optimized result into instructions for controlling the imaging device for carrying out a scan based on the localizer image.

According to a further embodiment, the invention relates to a control unit for an imaging device, wherein the control unit is intended for carrying out at least one embodiment of the above-described method.

According to a further embodiment, an imaging device includes the control unit.

The above-described inventive embodiments of the method can also be configured as a computer program product with a computer program, the computer being caused to carry out embodiments of the inventive method described above when the computer program is executed on the computer or on a processor of the computer.

An embodiment is further directed to a computer program with computer program code for carrying out all the method steps of at least one embodiment of the method claimed or described above when the computer program is executed on the computer or on a device (e.g. MR device). The computer program can also be stored on a machine-readable storage medium.

An alternative embodiment is directed to a storage medium which is intended for storing the computer-implemented method described above and is readable by a computer.

It is also within the scope of the invention that not all the steps of the method necessarily have to be executed on the same computer unit but can be carried out on different computer units or on another device (e.g. an MR device). The sequence of method steps can also be varied, if required.

The concepts used in the context of this invention will now be defined in greater detail.

### BRIEF DESCRIPTION OF THE DRAWINGS

Exemplary embodiments, which should be understood as not being restrictive, will now be described together with the features and further advantages thereof, making reference to the drawings, in which:

FIG. 1 is an overview of modules for carrying out the control method according to the invention according to a preferred embodiment of the present invention,

FIG. 2 is a flow diagram of a method in a training phase according to a preferred embodiment of the invention,

FIG. 3 is a flow diagram of a method in a control phase according to a preferred embodiment of the invention,

FIG. 4 is a schematic representation of a parallel re-training and

FIG. 5 is a schematic representation of a retrospective re-training.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Methods discussed below, some of which are illustrated by the flow charts, may be implemented by hardware, software, firmware, middleware, microcode, hardware description languages, or any combination thereof. When implemented in software, firmware, middleware or microcode, the program code or code segments to perform the necessary tasks will be stored in a machine or computer readable medium such as a storage medium or non-transitory computer readable medium. A processor(s) will perform the necessary tasks.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the following description, illustrative embodiments may be described with reference to acts and symbolic representations of operations (e.g., in the form of flowcharts) that may be implemented as program modules or functional processes include routines, programs, objects, components, data structures, etc., that perform particular tasks or implement particular abstract data types and may be implemented using existing hardware at existing network elements. Such existing hardware may include one or more Central Processing Units (CPUs), digital signal processors (DSPs), application-specific-integrated-circuits, field programmable gate arrays (FPGAs) computers or the like.

Note also that the software implemented aspects of the example embodiments may be typically encoded on some form of program storage medium or implemented over some type of transmission medium. The program storage medium (e.g., non-transitory storage medium) may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The example embodiments not limited by these aspects of any given implementation.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

In a preferred embodiment, the imaging device is a magnetic resonance device which can be operated with different recording techniques and parameters (e.g. different measuring sequences). However, the principle of embodiments of the invention is not fundamentally restricted to MR systems, but can be applied to other image processing algorithms which are based on particular training data and which are now to be applied to images that have been acquired with other contrast levels (or other recording techniques). Other embodiments can therefore relate to other imaging devices, for example, positron emission tomography devices, ultrasonic devices, computed tomography devices, etc.

Each image processing procedure (e.g. the automatic alignment process) is based on training data which are referred to in the following as a TRAIN image data set. The TRAIN image data set is characterized by the use of a particular “first” recording technique. The expression “recording technique” relates inter alia to a particular first MR pulse sequence, a first contrast level and/or further first recording parameters. The TRAIN image data set is therefore characterized by images which have been acquired with the first recording technique.

As distinct therefrom, the localizer image has been acquired or is in future to be acquired with another, second recording technique. The localizer image which is to be used for controlling the actual MR scan can therefore have been acquired with a second contrast and/or with a different MR pulse sequence than the TRAIN image data set. An unchanged use of the automatic alignment algorithm would therefore lead to errors. This is the starting point of the invention and it proposes a mechanism to make the results of the training for the TRAIN image data set usable also for other images that have been captured with another recording technique.

According to a first embodiment of the invention, the localizer image is identical to the at least one second image. According to an alternative embodiment, a matching module is provided which is intended to determine or select from the training image data set at least one second image which is associated with the localizer image. “Associated” indicates a correspondence according to an association rule which can be set in advance. The association rule can detect an identical second image for each localizer image. Alternatively, the association rule can detect, for each localizer image, a similar second image which has been acquired with a similar recording technique, particularly with the same or a similar contrast level.

The training image data set or TRAIN image data set can be unimodal and therefore based on images which have all been acquired from the same imaging device (e.g. MR) or it can be multimodal and therefore based on images which have been acquired with different imaging devices (e.g. CT, US, PET, MR). The TRAIN image data set can, in one embodiment of the invention, have been acquired from the same modality as the localizer image. Alternatively, the TRAIN image data set can also have been acquired from another modality (not MR). In particular embodiments, the images can originate, with the first recording technique and the second recording technique (with the respectively different contrast levels), from different imaging devices. In these cases, the image data are placed in relation to one another wherein image registration algorithms are applied to the image data sets to create a joint reference framework.

The localizer image and the image data sets of the TRAIN image data set can also originate from different studies. In this case, also, registration algorithms are used to correlate the respective image data.

The image processing procedure typically involves computer-implemented processes which are used in the context of the control of an imaging device. An image processing procedure can thus be a CAD algorithm (computer-aided diagnosis or pattern recognition process) which is based on particular training data. The image processing procedure can be, in particular, an automatic alignment process. In existing modern MR devices, for example, an automatic alignment procedure is provided, so that in, for example, a knee examination, the user can only confirm the data set automatically suggested by the automatic alignment algorithm in order to adjust the MR device, comprising a volume segment (field of view) and the slice orientation as well as the required spacing dimensions. The parameters for the proposed MR scan are therefore automatically suggested to him without his having to make the relevant settings by hand from a low resolution overview image. Regardless of the respective positioning of the patient, the MR system with an automatic alignment technique automatically carries out a reproducible slice positioning and thereby simplifies and accelerates the control of the planned MR investigation. The automatically executed automatic alignment procedure is advantageously independent of the respective coil setting or the respectively used measuring technique. A further advantage of the use of automatic alignment techniques is found therein that for many scans, the same positioning criteria are applied and thus good reproducibility of the measuring results can be made available for each patient, as well as across all patients. However, the invention is not restricted to the automatic alignment algorithm as an image processing procedure, but can also relate to more complex CAD uses which are designed, for example, specifically to segment and identify and/or digitally process a particular organ or bone or body structure. Other image processing procedures are also based on learned CAD knowledge, which can find expression, inter alia, in the positioning of landmarks or in the segmentation of anatomical structures.

With the automatic alignment technique, a prior recording, known as a “localizer image”, or an image data set is analyzed with the aid of validated training data in order to control the subsequent, actual MR scan. “Control” in this context means the setting and selection of the various control parameters for the MR scan, for example, the selection of the measuring sequence with position encoding and particular settings relating to image formation. With the aid of the automatic alignment algorithm, these settings can be calculated entirely automatically and must only be confirmed by the user.

At least one embodiment of the invention essentially proposes that initially the automatic alignment process or another image processing procedure is used on part of the TRAIN image data set to generate an interim result therefrom. However, the interim result must be further processed, since the localizer image is present at a different contrast level or has been acquired with a different recording technique than the training image data. Therefore, the calculated interim result is transferred to the respective localizer image in order to calculate a result. Then the result of an optimization method is supplied in order to improve the quality of the result. The optimized result thus provided is subsequently converted into instructions for controlling the MR device for carrying out the scan based on the localizer image.

The optimization method involves statistical methods which can comprise statistical training. The optimization method can also comprise a calculation of confidence intervals. Furthermore, anatomical limitations (e.g. due to the size and position of the organs, etc.) and/or context information (e.g. laboratory measurement values or other data sets which can be retrieved from medical databases, for example) can also be taken into account.

With at least one embodiment of the invention, it is possible to transfer CAD knowledge learned earlier with which the image processing procedure has been trained to almost any desired images (for example, localizer images which have been acquired with a different contrast). Advantageously, in this way, the control method according to the invention can also be applied if the second recording technique (which has been applied in the capture of the localizer image) and the first recording technique (which has been applied in the acquisition of the training image data) differ from one another.

The training image data set (which is also denoted in the following synonymously as the TRAIN image data set) typically comprises annotations. The annotations can include, for example, of inserted landmarks to identify body structures, organs or other image features. The interim result is obtained in that the image processing procedure is applied to the TRAIN image data set. Since, however, the recording technique of the TRAIN image data set does not match the recording technique of the current localizer image, the interim result cannot yet be used for controlling the imaging device based on the localizer image. Initially, the interim result must be transferred to the localizer image. In other words, the trained knowledge must be transferred from the existing image processing procedure to other contrast levels.

According to at least one embodiment of the invention, this is automatically enabled in that, over a particular time span, a particular number of patients are examined both with the first recording technique (e.g. first contrast level) and with the second recording technique (e.g. second contrast level) in parallel, in order to be able to provide an annotated training data set. The annotated image data set is based on the use of the image processing procedure on “old” image data sets (i.e. on image data sets which have been acquired with the old or with the first recording technique) for each individual case or for each individual image data set. The training data annotated in this way or the interim result is used to train the algorithm or the image processing procedure to the new contrast level and thereby in general to the “new” or second recording technique.

In order to increase the quality of the control system overall or of the transfer procedure, in an advantageous embodiment of the invention, it can be provided that during the transfer of the calculated interim result, a quality identifier for the existing CAD algorithm which was used on image data with the old contrast level is taken into account. Statistical methods can be used herein so that, for example, only those interim results are used for the transfer which fulfill pre-defined safety criteria, that is, enable an interim result in a particular quality level. A setting can be made so that only selected annotated data sets are taken into account for the training of the new contrast level. It can thereby be ensured that only those annotations which have been validated as safe or correct are taken into account for the training of the new contrast level. The validation can be carried out in a preparation phase. The validation can be carried out, for example, through manual confirmation by a user. It should be noted herein that the manual verification by a user is carried out in a preparation phase during training of the algorithm. The transfer of the CAD knowledge to image data sets which have been acquired with another recording technique can take place entirely in the background and requires no user interactions.

The method according to at least one embodiment of the invention requires only that in a preparatory phase (training phase) which precedes the actual use of the method for controlling the MR device, different recording techniques are applied during the same scan. Thus, the image data are acquired with the first recording technique and with at least one further recording technique, in particular the second recording technique. Thus, the data acquisition for the first recording technique and the second recording technique take place during the same scan of the patient (without the patient moving), so that the same reference frame can be used for the respective images or for the image processing thereof. The first recording technique and the at least second recording technique are effectively applied in parallel. “Parallel” in this context means that during an investigation, different recording techniques are applied. Naturally, these recording techniques can also be carried out in practice sequentially. Thus, for example, initially a first MR scan is carried out at a first contrast level and then a second MR scan at a second contrast level without the patient moving, so that the same reference frame can be maintained. Naturally, further recording techniques can also be applied in order to generate a plurality of second images. These data sets are preferably stored in a central database.

Naturally, more than two different recording techniques can also be put into use in parallel during the training phase. For example, it is possible, during one and the same scan of a patient to apply and record five different contrast levels and three different pulse sequences. If, during the later execution of the method, the localizer image is then available in one of the contrast levels or pulse sequences executed, then the collected training knowledge can be transferred automatically to the respective recording technique. This is achieved in that the matching module selects the respective corresponding second image from the training image data set and associates it with the localizer image.

The quality can be increased, for example, in that an additional validation is carried out on the transferred CAD knowledge to the localizer image. The user can herein also confirm or reject selected transfer processes in a targeted manner, in order to improve the re-training of the image processing procedure to the further image data set in the further recording technique.

In a preferred embodiment, the transfer comprises the transferring of anatomical landmarks automatically captured in the TRAIN image data set to the localizer image. The annotation data which have been automatically set in the TRAIN image data set are automatically transferred to the localizer image. A recording technique is characterized inter alia through the respective contrast levels set with possible differences in the slice thickness, in the pixel size and/or in the field of view.

Depending on the embodiment of the invention, different image processing procedures can be applied, for example, an automatic alignment algorithm or algorithms for automatic pattern recognition in the image processing.

According to at least one embodiment of the invention, the training phase is characterized in that the training image data set is recorded not only in one recording technique, but in at least one second recording technique, wherein a constant frame of reference (with regard to the positioning) must exist (that is, without a change in the position of the patient).

According to an alternative embodiment, the different recording techniques are carried out for capturing the TRAIN image data set in different scans. However, the common reference framework is guaranteed in that registration methods are applied in both the scans in order to be able to place the two image data sets in relation to one another. The registration methods used therein are known from the prior art. In this regard, reference is made to the granted patent DE 102011083766 B4 which concerns a method and a device for overlaying an X-ray image with a projection image from a 3-D volume data set of a rotational angiogram.

If the different recording techniques during the capture of the images for the TRAIN image data set are not to be recorded in a common scan, then in an alternative embodiment of the invention, it can be provided that they are retrieved from a database retrospectively.

In principle, it can be taken that the algorithm is trained in that a particular training data set of images which have been acquired in a first recording technique is used.

Two embodiment forms are provided for the invention:

1. a prospective or parallel re-training and

2. a retrospective re-training.

The prospective or parallel re-training relates thereto that in a preceding training phase, a different type of data recording of the MR image data is carried out. While the patient is suitably supported for an MR investigation, in the same study (therefore without moving the patient), images are recorded with a first and at least one second recording technique (that is, for example, with a first contrast, with a second, different contrast and with a third, different contrast). It is therefore expressly within the scope of the invention to use not only one second recording technique, but a plurality of second recording techniques. The second recording technique differs from the first recording technique. Thus, the images which have been recorded in the training phase with the first recording technique and with the second recording technique or with the other recording techniques can be correlated to one another. This is required for the subsequent image processing procedure and the annotation of the image data.

In a first, parallel embodiment of the invention, a further image data set is acquired wherein the first recording technique (e.g. first contrast level) and the second recording technique (e.g. other contrast level) have both been applied. The algorithm is then applied to all the images with the first recording technique (e.g. old contrast) in order to obtain an interim result. All or selected interim results are then transferred to the other image data sets, particularly to the second image which the matching module has associated with the localizer image and which has been acquired with the second recording technique (with the new contrast). Then, the images with the second recording technique (e.g. with the new contrast) are used in order to train the algorithm again (re-TRAIN). The re-trained algorithm is then used for further processing of the image studies acquired with the second recording technique (e.g. with the new contrast).

In the retrospective embodiment, a central database is provided which can be operated, for example, in a cloud and is accessible by different organizations (hospitals, medical practices, etc.). In the database, image data are stored which can also be used as training image data sets. The image data sets comprise, for a patient scanned in one study, images which have been acquired with a first recording technique and images which have been acquired at least with another, second recording technique. From this database, by means of a suitable database access, a quantity of image data are obtained which have been captured both with the first recording technique and also with the at least one second recording technique or with further second recording techniques. This quantity of image data sets is used for the re-training and is read into the control unit.

In the second retrospective embodiment of the invention, no acquisition phase for image data sets in the first and second recording technique takes place, but rather access is made to a database in which image studies are stored. In particular, a study of this type is sought which includes images that have been acquired both in the first and the second recording technique in the same study. The algorithm is then applied to all the images captured with the first recording technique (e.g. the old contrast level). Subsequently, all or selected interim results are applied indirectly to the localizer image and directly to (a plurality of) second images which have been acquired with the second recording technique (e.g. with the new contrast level). Then, the second images captured with the new contrast level (second recording technique) are used in order to train the algorithm again (re-training). The re-trained algorithm is also subsequently used in this embodiment in order further to process the image studies with the new contrast level.

Typically, a localizer image is used as the input or as an input variable for an automatic alignment algorithm. However, it is also possible to use a plurality of localizer images and thereby to provide a localizer image data set. This is particularly advantageous if the method is not applied for an automatic alignment algorithm, but for other pattern recognition algorithms.

According to one embodiment, the TRAIN image data set is characterized in that it contains automatically generated landmarks with which the image data set is annotated, for example, in order to identify anatomical structures in the image data set. The annotations can be converted into instructions in order to be able to carry out a subsequent scan with the imaging device focused on the particular anatomical structure which has been annotated in the TRAIN image data set.

The instructions for controlling the MR device or other imaging devices comprise commands for setting a field of view (FoV), a slice positioning, a position of the slices to be recorded, a setting regarding the number of layers and/or the thickness of the slices for a complete or optimized coverage of the field of view and additional saturation regions which are, in principle, often used for MR imaging in order to suppress artifacts. These settings serve to optimize the subsequent MR scan for the respective anatomical structure.

In order to prevent errors in the transfer of the CAD knowledge or the training knowledge and in particular to be able to preclude the transference of faulty annotations which are to be transferred from the old recording technique to the new recording technique, a variety of different methods can be used. The different methods can be carried out individually or in combination with one another. These methods comprise, as previously mentioned above, statistical methods for applying a confidence interval for the training knowledge or the annotated landmarks. The confidence interval can be improved by means of suitable learning algorithms. Furthermore, anatomical limitations can be taken into account. The anatomical limitations relate, for example, to the knowledge regarding the siting and position of particular landmarks in organs or body structures in the human body. An automatic algorithm can be carried out to check anatomical consistency.

Furthermore, context information can be taken into account in the validation of the checking. For example, different selection criteria can be applied in the transfer. It is, for example, possible to take account only of complete studies in which no corrections and/or no user interactions took place in relation to the images that have been captured with the first recording technique (e.g. at the old contrast level). Furthermore, studies in which a patient movement or other disturbances have been recorded during the MR scan can also be excluded.

At least one embodiment of the invention is further directed to a control unit for an imaging device, for example, an MR device wherein the control unit is intended for carrying out the above-described method.

The control unit, in at least one embodiment, comprises a training unit which is intended for training and re-training the training data set and/or the TRAIN image data set.

The control unit, in at least one embodiment, further comprises an interface to the imaging device, by means of which each image data set comprising the TRAIN image data set and the localizer image data set can be read in.

In principle, a plurality of different embodiments can be implemented. It is thus possible that the control unit is integrated into the imaging device as a separate module. It is also possible to provide the control unit as a separate module which exchanges data with the MR system via a data connection.

The control unit, in at least one embodiment, also comprises an image processing module which is intended for carrying out the image processing procedure (e.g. automatic alignment process).

Furthermore, the control unit, in at least one embodiment, comprises a processor which is intended for processing the image data, as described above in relation to the method. The processor serves, in particular, for transferring the interim result to the localizer image and for optimizing the result.

The control unit, in at least one embodiment, also comprises an instruction unit which comprises an interface to the MR device and is intended to control the MR device for the subsequent scan. The instruction unit serves to convert the optimized result into instructions for controlling the imaging device for carrying out the scan based on the localizer image.

The control unit can also comprise a matching module which is intended for carrying out an association rule and for determining an association between the localizer image and a second image.

In another embodiment, an imaging device with a control unit as described above is disclosed.

The control unit can also exchange data with a central database and optionally also a local memory store in which interim results and/or results can be stored. It is also possible to store all or selected image data sets locally in the control unit.

The above-described inventive embodiments of the method can also be configured as a computer program product with a computer program, the computer being caused to carry out embodiments of the inventive method described above when the computer program is executed on the computer or on a processor of the computer.

An embodiment is further directed to a computer program with computer program code for carrying out all the method steps of at least one embodiment of the method claimed or described above when the computer program is executed on the computer or on a device (e.g. MR device). The computer program can also be stored on a machine-readable storage medium.

An alternative embodiment is directed to a storage medium which is intended for storing the computer-implemented method described above and is readable by a computer.

It is also within the scope of the invention that not all the steps of the method necessarily have to be executed on the same computer unit but can be carried out on different computer units or on another device (e.g. an MR device). The sequence of method steps can also be varied, if required.

Furthermore, it is possible for individual portions of the above-described method to be implemented in a commercially saleable unit and for the remaining components to be implemented in another saleable unit—effectively as a distributed system.

The invention will now be described in greater detail making reference to the drawings and actual example embodiments.

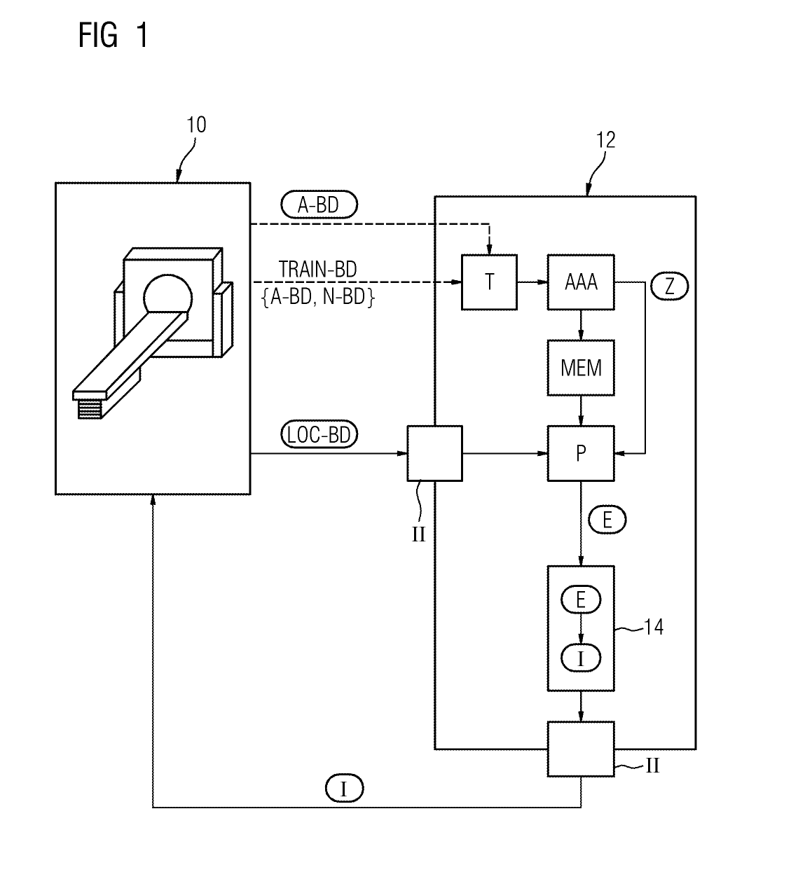

In FIG. 1, the schematic configuration of the system according to the invention is described in greater detail, comprising an imaging device which is configured in this case as an MR device 10, and a control unit 12. The MR device 10 and the control unit 12 exchange data with one another. Alternatively, the control unit 12 can also be integrated directly as a module into the MR device 10. The control unit 12 serves to output instructions I which serve to control the MR device for a subsequent MR scan.

The control unit 12 comprises an image processing module AAA, which is designed for carrying out an image processing algorithm. In the preferred embodiment, the image processing algorithm is an automatic alignment algorithm which belongs to the group of ALPHA algorithms which are used, inter alia, for automatic alignment for knee MR examinations, spine examinations, shoulder examinations, hip and breast examinations. In addition, the automatic alignment algorithms of the ALPHA group have been used for cardiac catheter examinations and in MR-supported carotid artery scans.

However, the problem lies therein that the ALPHA algorithms have only been trained with particular image data. The image data have been recorded in a particular contrast of an original training image data set. Therefore, the existing ALPHA algorithm cannot be used at other MR contrast levels.

In principle, a localizer image LOC-BD which has been recorded by the MR device 10 serves to derive and calculate the corresponding parameters in order to be able to set the MR device 10 for the subsequent scan. Based on the algorithm of the image processing module AAA, for example, commands for setting the field of view, FoV, for slice positioning, for defining the number, the position and the thickness of the slice to be recorded should be set. These settings are encoded in a digital data set which is transferred by means of a suitable output interface OI from the control unit 12 by means of calculated instructions I to the MR device 10 for execution. The instructions I serve to control pre-determined actuators, motors and other technical modules for controlling the MR device 10.

The automatic alignment algorithm of the image processing module AAA is trained in a chronologically preceding training phase with image data sets which have been recorded, for example, by the MR device 10. The training per se is not a part of the present application but is briefly described here for the sake of completeness in order to make clear the context of the solution according to the invention. The training of the automatic alignment algorithm is based on “first images A-BD” which have been acquired in a first recording technique. According to one embodiment, the first recording technique relates to a first contrast level. Other embodiments provide other parameters of the MR scan, for example, other pulse sequences (T1-weighted scan, T2-weighted scan, etc.). The training is carried out in that, based on the relevant training image data set, annotations are included in the image data and these are evaluated in a subsequent evaluation process with regard to their grade and quality. The evaluation process can be carried out automatically, semi-automatically or manually. Depending on how good the annotations are, these are inserted into the image data and are used iteratively for further training cycles.

Qualitatively inferior annotations are rejected. The purpose of an automatic alignment algorithm of the ALPHA group can therefore be annotated image data which identify particular anatomical structures (vessels, organs, etc.) which, in turn, are automatically converted in subsequent processing steps into instructions I for controlling the actual MR scan 10. For this purpose, an overview image or a localizer image data set LOC-BD is recorded which is typically captured in low resolution once the patient has already been placed in the MR device 10. The localizer image comprises a sequence of image data sets which are analyzed to calculate the instructions I so that the MR device 10 can be specifically set to the relevant anatomical circumstances of the organ to be investigated.

In the prior art, it was only possible to use the automatic alignment function for such localizer images LOC-BD which had been captured with the same recording technique as the training data with which the automatic alignment algorithm had been trained. However, if the localizer image had been acquired with another recording technique, it was not possible to use the automatic alignment algorithm. This problem is solved by the present invention in that the control unit 12 is extended with additional modules.

The control unit 12 thus also comprises a processor P. The processor P serves to record the localizer image data set LOC-BD which has been captured by means of an input interface II. Furthermore, the processor P serves to receive an interim result Z which has been provided by the image processing module AAA. Furthermore, the processor P serves to transfer the calculated interim result Z to the localizer image LOC-BD for calculating a result E which is output and can also be converted directly by the processor P into instructions I. The instructions I are then passed on via an output interface OI to the MR system 10 for the purpose of control.

As indicated in FIG. 1, the control method is fundamentally divided into two time phases, specifically a training phase and a control phase. In the training phase, the automatic alignment algorithm of the image processing module AAA is trained with training data which can originate from an MR device 10. This must not necessarily be the same MR device 10 which also serves to record the localizer image LOC-BD. It may be another device of the same or another modality (ultrasound, CT, etc.). The data which are passed in the training phase from the MR system 10 to the control unit 12 are shown dashed in FIG. 1. This involves a first image A-BD, which has been acquired with a first recording technique (e.g. with a first contrast level), and a TRAIN image data set TRAIN-BD. The TRAIN image data set comprises at least one second image N-BD for each first image A-BD. The second image N-BD is characterized in that it has been acquired with another, second recording technique which differs from the first recording technique (for the first image), but originates from the same or an assignable scan or study (for a patient and an organ-specific examination of the patient). It naturally also lies within the framework of the invention that the training image data set TRAIN-BD consists not only of a first image (or first image data set) and a second image (or second image data set), but comprises a plurality of second image data sets each acquired in different contrast levels or with different recording techniques (as distinct from the first image with a “new” recording technique). The first image A-BD and the training image data set TRAIN-BD with the second image N-BD is fed to a training unit T which serves for training the automatic alignment algorithm in the training phase. The result is annotated images which serve as the interim result Z of the further processing and are output by the image processing module AAA.

For the actual control of a planned MR scan, in the immediate preparation for the actual scan, the patient is placed in the MR device 10 and a localizer image LOC-BD is recorded. This takes place in the second phase, specifically in the control phase and is therefore indicated in FIG. 1 with a solid arrow which is continued via the input interface II to the control unit 12. The processor P then receives the localizer image data set LOC-BD and accesses the interim result Z, which is provided by the image processing module AAA and is now intended to transfer the calculated interim result Z to the read-in localizer image LOC-BD which is associated with the second image, in order to be able to provide a result E. The result E can then be fed to an optimization method which comprises the calculation of confidence intervals and other statistical methods. Furthermore, the optimization method can take account of anatomical limitations and context information of the respective MR scan or of the patient (e.g. size and position of the patient). Following use of the optimization method on the result E, the result E is directly converted into instructions I, which are passed on to the MR device 10 for control. This is indicated in FIG. 1 with the arrow which is directed from the control unit 12 to the MR device 10.

According to one embodiment, it is provided that the functionality of the processor P is subdivided among different components. Thus, in particular, the functionalities which serve to convert the optimized result E into instructions for controlling the MR device 10 are transferred into an instruction unit 14. The instruction unit 14 calculates the instructions I from the optimized result E and passes them via the output interface OI to the MR device 10. Alternatively, however, the instruction unit 14 can also be integrated into the processor P so that only one common component is provided.

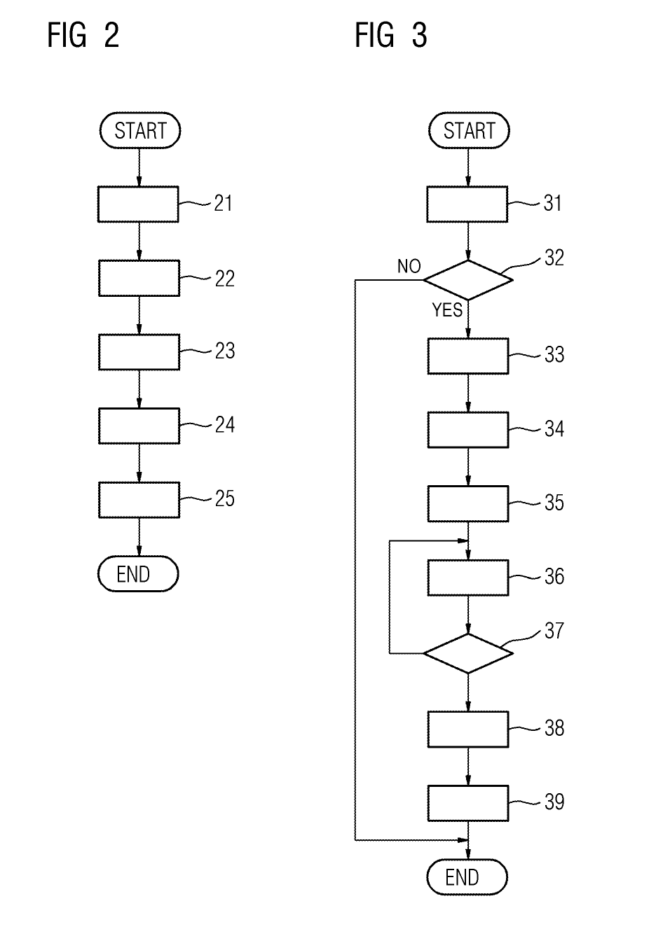

FIG. 2 shows a possible sequence of a training phase according to a preferred embodiment of the invention.

Following the start, in step 21, the recording of the training image data set TRAIN-BD takes place on the MR device 10 (or on other imaging devices). The TRAIN image data set TRAIN-BD comprises a first image data set A-BD and at least one further second image data set N-BD with a new contrast. As mentioned above, it is also possible that the training image data set TRAIN-BD comprises further second image data sets N-BD which however have also been acquired with a different recording technique (e.g. newer, different contrast level, new, different pulse sequence, etc.), like the first image data A-BD which have been captured with the first, or old, contrast level.

In step 22, the automatic alignment algorithm or an algorithm of the ALPHA group is applied to the first image data set A-BD. What is essential is that for each first image data set A-BD, at least one second image data set N-BD has been acquired in the same scan and is present and can thus be unambiguously associated with the first image data set A-BD.

In step 23, the result, in particular the inserted annotations, is evaluated.

In step 24, the automatic alignment algorithm is applied again, based on the evaluated results. This method can be repeated iteratively.

In step 25, an automatic alignment algorithm or an image processing procedure which has been trained with the first image data set A-BD can be provided as the result. Once the training phase is completed, the image processing procedure can be provided as a validated process in the image processing module AAA.

FIG. 3 shows a control phase which is carried out, from the time standpoint, retrospectively once it has been possible to complete the training phase entirely.

Following the start of the control phase, in step 31, the localizer image data set LOC-BD is captured. In step 32, a check is carried out of whether the localizer image data set LOC-BD has been acquired in the same recording technique as the first image data set A-BD with which the automatic alignment algorithm of the image processing module AAA has been trained. If it is ascertained that the first recording technique of the first image data set A-BD matches the second recording technique of the localizer image LOC-BD, the method for the automatic alignment procedure known in the prior art can be carried out. Otherwise, the method branches into steps 33 to 39.

Next, in step 33, the automatic alignment algorithm is applied to the first image data set A-BD of the training image data set TRAIN-BD.

In step 34, an interim result Z is generated.

In step 35, the interim result Z generated is indirectly transferred to the recorded localizer image LOC-BD. This is carried out in that the annotations from the first image are transferred to the second image which is associated with the localizer image.

In step 36, a result E is generated.

In step 37, the result E is possibly optimized in a multi-step method. The optimization can be carried out according to statistical algorithms, anatomical conditions or context conditions. The optimization can also be performed iteratively, so that a generation of a result E is repeated.

Subsequently, in step 38, the result E is converted into instructions I.

Finally, in step 39, the MR unit 10 is controlled with the generated instructions I.

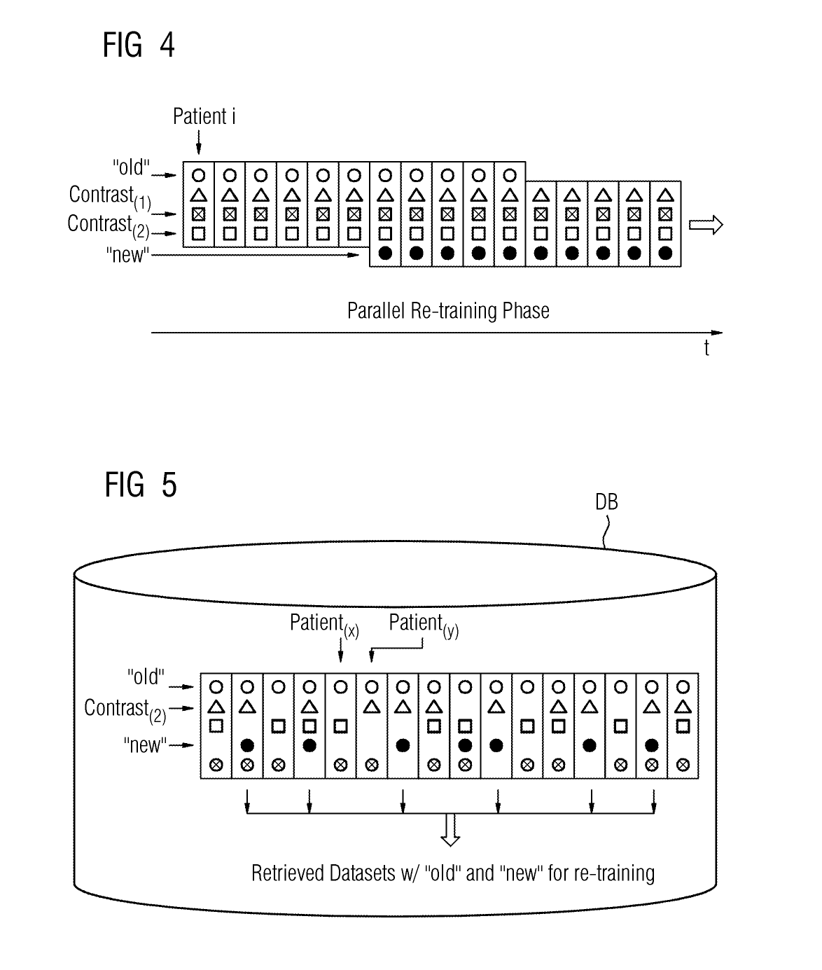

In principle, the invention can be implemented in two different embodiments, specifically in a parallel re-training process which will now be described in greater detail making reference to FIG. 4 and in a retrospective re-training process which will then be described in greater detail making reference to FIG. 5.

FIG. 4 shows schematically the chronological sequence of a training process for an image processing algorithm of the image processing module AAA wherein different recording techniques (e.g. contrast levels) are used in parallel and thus during an MR scan. As FIG. 4 shows, during a particular time period, an old contrast level (first recording technique) and simultaneously, a second contrast level (in general: second recording technique) are expressly acquired together for a set of patient studies in order to enable the later transfer of the algorithmic knowledge to images of the new contrast level. As shown in FIG. 4, in the seventh to eleventh time phase, in parallel with the old contrast level, a scan is also carried out at the new contrast level.

FIG. 5 shows the retrospective re-training. In this case, a large centrally administered database DB is provided which can be connected in, for example, as a virtualization of the control unit 12. The database DB can also be accessible from the control unit 12 via a corresponding network connection. The database DB serves to store patient studies which have been acquired with different recording techniques (e.g. different contrast levels). By means of suitable database codes, the studies which have been carried out at the old contrast level as well as at the new contrast level can easily be determined and these are then used for re-training of the algorithm.

The retrospective re-training offers the advantage that easier implementation by the final user is enabled. A central administration unit can administer the database DB which can be operated, for example, as a cloud database and in which the greatest possible number of MR studies is stored. The studies herein comprise image data sets which have been recorded with a first recording technique and a second recording technique and further second recording techniques. This preferably involves high resolution MR scans.

Due to the constantly changing requirement conditions for the performance of MR examinations, new requirements are being placed over and again regarding the recording techniques. For example, at present, ever higher requirements are being made concerning the perceptible and disturbing sounds during an MR examination and there is therefore a need for the execution of “quiet” MR examinations (“quiet scans”). Particular recording techniques which achieve this aim must therefore be selected here.

Embodiments of the invention therefore makes it possible for the localizer image LOC-BD recorded in the control phase to have been acquired with a different recording technique than the image data with which the algorithm was originally trained. In a further embodiment, although the localizer image LOC-BD is acquired with the same recording technique as the images with which the automatic alignment algorithm has been trained, requirements are placed on the scan to be performed subsequently so that the interim result Z must be transferred from the first image data A-BD to other image data. In this case, also, the method according to the invention can be used for transferring the annotations to the localizer image LOC-BD. One advantage of the retrospective solution is found therein that the database DB of different medical devices can be operated worldwide so that it can be ensured that a large number of different image data sets are present which are present with the second recording technique (the new contrast level) for all the respectively desired organs or body parts (e.g. knee, femoral neck, patella, spinal column, etc.).

According to a preferred development of the invention, a further revision module (not shown in the figures) is provided which serves to prevent false interim results Z (which have been determined at the old contrast level) from being transferred to the new contrast level. In order to ensure this reliably, the module accesses the optimization method.

It must also be ensured that the re-training process is successful. For this purpose, a self-assessment module (not shown in the drawings) based on statistical methods is provided. For this purpose, the method can be accessed on a “leave-n-out-cross-validation” approach and a plurality of iterations of re-training can be applied to each new data set. Then, for each re-training, a test is carried out on the “leave-out” data set and the deviations of the test results are recorded in relation to model values (ground truth). Thereupon, the data sets with the greatest deviations or with deviations that exceed a pre-defined threshold can be erased from the new training data set. The re-training can then be carried out with the training data set changed (reduced) in this way.

Finally, it should be noted that the description of the invention and the exemplary embodiments should not be seen as in any way restrictive with regard to a particular physical realization of the invention. For a person skilled in the art, it is obvious that the invention can be realized partially or entirely in software and/or hardware and/or distributed over a plurality of physical products—particularly also computer program products.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for controlling an imaging device by using an image processing procedure on at least one localizer image recorded by the imaging device, the method comprising:
providing an image processing procedure, trained in a training phase with first images of a TRAIN image data set, the first images having been acquired using a first recording technique and the TRAIN image data set, for each first image, includes at least one second image, the at least one second image having been acquired with a different, second recording technique;
providing the localizer image for use of the image processing procedure, the localizer image having been acquired with the second recording technique;
applying the image processing procedure to the first image of the TRAIN image data set for calculating an interim result;
transferring the calculated interim result to the localizer image associated with the second image to calculate a result;
using an optimization method on the result; and
converting the optimized result into instructions for controlling the imaging device for carrying out a scan based on the localizer image.

2. The method of claim 1, wherein, in the previous training phase, in parallel and during the same scan, images are acquired with the imaging device using the first and the second recording technique.

3. The method of claim 1, wherein the first and the second recording technique are different.

4. The method of claim 1, wherein the optimization method is carried out iteratively on the localizer image and serves for training the image processing procedure to the localizer image.

5. The method of claim 1, wherein the optimization method takes account of a calculation of at least one of confidence intervals, anatomical limitations and context information.

6. The method of claim 1, wherein the optimization method comprises a statistical training process.

7. The method of claim 1, wherein the transfer comprises an automatic transfer of anatomical landmarks and annotations automatically recorded in the respective first image of the TRAIN image data set into the localizer image.

8. The method of claim 1, wherein the first recording technique comprises a first contrast level and the second recording technique comprises a second contrast level, which differs from the first contrast level.

9. The method of claim 1, wherein the image processing procedure comprises at least one of an automatic alignment algorithm and an algorithm for automatic pattern recognition.

10. The method of claim 1, wherein the localizer image and the TRAIN image data set are recorded in a separate scan of the imaging device.

11. The method of claim 1, wherein the first image and the second image of the TRAIN image data set have been recorded from different scans, but have a common reference framework.

12. The method of claim 1, wherein the localizer image and the TRAIN image data set are prospectively recorded in a training phase.

13. The method of claim 1, wherein the localizer image and the TRAIN image data set are retrospectively read out of a database.

14. The method of claim 1, wherein the image processing procedure automatically annotates a respective image, via landmarks, to identify anatomical structures in the image which are convertible into instructions to be able to carry out a scan via the imaging device focused on the relevant anatomical structure.

15. The method of claim 1, wherein the instructions for controlling the imaging device comprise commands for setting a field of view, at least one of a slice positioning and a position, thickness and number of the slices to be recorded in order to scan a desired anatomical structure.

16. The method of claim 1, wherein the first image and the second image of the TRAIN image data set have been recorded by different medical imaging devices and have been referenced by way of a registration algorithm.

17. A control unit for an imaging device, the control unit, configured to carry out the method of claim 1, comprising:
a training unit, configured to train the TRAIN image data set;
an interface to the imaging device, configured to provide the TRAIN image data set and a localizer image;
an image processing module, configured to carry out the image processing procedure;
a processor, configured to transfer the interim result to the localizer image and to optimize the result;
an instruction unit, configured to convert the optimized result into instructions for controlling the imaging device to carry out a scan based on the localizer image.

18. An imaging device comprising:
the control unit of claim 19.

19. The method of claim 2, wherein the first and the second recording technique are different.

20. The method of claim 2, wherein the first recording technique comprises a first contrast level and the second recording technique comprises a second contrast level, which differs from the first contrast level.
